(19) United States
(12) Patent Application Publication
(10) Pub. No.: US 2011/0144728 A1
(43) Pub. Date: Jun. 16, 2011

(54) TANNING LAMP ARRANGEMENT

(76) Inventors: Attila Soltész-Nagy, Miskolc (HU); Attila Sándor Soltész-Nagy, Miskolc (HU); Tamás Soltész-Nagy, Miskolc (HU)

(21) Appl. No.: 13/058,001

(22) PCT Filed: Aug. 5, 2009
(86) PCT No.: PCT/HU09/00071

§ 371 Date: Feb. 17, 2011

(30) Foreign Application Priority Data
Aug. 8, 2008 (HU) P0800505

(51) Int. Cl. A61N 5/06

(52) U.S. Cl. 607/94

(57) ABSTRACT

Tanning lamp arrangement, especially for suntanning the skin surface of the human body, having at least one UV light source (2) irradiating UV light with an appropriate intensity also in the UV-A and/or UV-B spectral ranges, and having optical elements reflecting the light of the light source(s), placed upon the side of the human body opponent to the light source(s). The tanning lamp arrangement, according to the invention has at least one light source arranged for the exposure of one side of the body, placed along a particular line, essentially parallel with the vertical axis of the body, and further the light reflecting optical elements are designed as a first light reflecting surface (1) producing an uniformly scattered light for the exposure of the body.

### TECHNICAL FIELD

The subject of the invention is a tanning lamp arrangement, especially for tanning of the skin of the human body, having at least one ultraviolet (UV) light source emitting UV light with an appropriate intensity also in the UV-A and/or UV-B spectral ranges, and having optical elements reflecting the light of the light source(s) placed upon the side of the human body, opponent to the light source(s).

### BACKGROUND ART

Arc light is considered to be one of our oldest artificial light sources, extensive, widespread use and known in the technical field. In 1849, it has already been discovered by Foucault that its spectrum is similar to our major natural light source, the Sun.

In accordance with a definition in the New Hungarian Encyclopedia, arc light is a light emitting electrical arc evolving between carbon rods or metal rods, supplied by electric current, contacted to each other, and then pulled apart. The arc light lamps are special light sources, still in use today, but rarely. The oldest type of them was functioning with carbon electrodes, its very intensive light made it suitable for them being applied in projectors, in stage illumination, too. Nevertheless, lot of difficulties have arisen in connection with the use of arc light, since its stability (steadyness of the plasma arc) was hard to secure, since the electrode tips made from graphite had been oxidized and consumed by burning, and therefore a continuous adjustment was necessary. (There is a process of oxidation and electrode loss in case of metal rods too, although to a less extent.) As a consequence of all these, a change came to the gas-filled discharge lamps, containing also a lighting plasma arc, and the use of the traditional arc light lamps has gradually been driven back, limited to the fields of application mentioned; these lamps, so to say, “went out of fashion”.

The ultraviolet (UV) light is an electromagnetic radiation of shorter wawelengths then the visible light. Normally it is divided according to wawelength as the UV-A (315-400 nm), the UV-B (315-400 nm), and the UV-C (100-280 nm) spectral ranges.

Considering their physiological effects, the UV-C and UV-B radiations imply serious and direct danger to the metabolism and functioning of epithelial tissue cells of the skin, occasionally giving rise to malignant and irreversible changes in them.

By using UV-A radiation, however—apart from the genetically determined sensitivity among some human types, or from individual hypersensitivities—these above mentioned harmful effects, were not observed to that extent, but pursuant to experiences—depending upon pigmentation of the skin—a uniform tanning, a gradual development of the desired skin colour can be achieved by means of the effect of this irradiation.

During the past two decades, scientific research has revealed that the ozone layer protecting the Earth surface from the harmful UV-irradiation has become thin and tapered off (so-called ozone layer hole). As a consequence, malignant skin lesions, skin diseases are more frequently occurring, and especially there where sunbathing outdoors used to be very popular, as, for example, in Australia. The dramatic surge in the melanoma (pigmented skin tumor) cases and its spread—taking into account the bad prognosis, the generally fatal outcome of this malady—has shocked the public and alarmed dermatologists. Since the research has determined the relation between the two phenomena, the sunbathing significantly declined. On the other hand, as the civilization needs are growing, people—especially the ladies—do not want to give up their advantageous, suntanned looking. It can be the explanation to the ever growing popularity of the solarium treatments in our days.

There is an enormous demand for reliable solarcosmetic services that people could make use of controlled conditions, minimizing the health risks, instead of sunbathing outdoors that becomes more and more dangerous.

A number of various light sources have been tried out and applied so far for these purposes, for example, fluorescent light tubes, more recently lasers, and several gas-filled discharge lamps, or discharge lamps with mercury vapor filling, while the latter resemble most to arc light lamps, regarding their operation principle.

According to the Hungarian patent HU 221 825, titled “Discharge lamp emitting UV radiation, namely solarium lamp” purpose of that invention is to provide discharge lamps, especially solarium lamps that produces high UV-A emission. (Apparently, the reason for it is the view widely held in phototherapy as well as in solarcosmetics, based on observations, that the uniform suntanning of the skin, without harmful side effects is mainly to be attributed to the part of the UV spectrum belonging to the UV-A range). The patent specification states “The radiation produced by the electric discharge in solarium lamps will be transformed with the participation of a phosphor to UV-A radiation capable of bronzing the skin.” Accordingly the task of the invention will be solved by a phosphor—of improved properties—in this case. Working of the lamp is based on photoluminescence; the spectrum (spectral distribution) of the light emitted by the phosphor is totally different from the exciting UV light.

The Hungarian patent HU 224 941, titled “Phototherapy apparatus”, describes an application of UV light beam, also using mercury discharge lamp as the light source; concave mirror reflecting part of the UV light beam, further condenser lens focusing the UV light beam, moreover an optical filter and the adjoining light transmitting system are parts forming the sophisticated device, the therapeutic application of which has however an enormously wide scale. An essential feature of the invention is the application of precisely dosed (exposure time, light intensity) UV light beams, concentrated to the given body parts, according to the illness to be cured.

As it is stated in the Hungarian patent application, P0402200, (Solarium equipped with light conductor) the application of a high power UV/A light source is expedient instead of the tubes recently in widespread use.

From the disclosure of the patent specification DE 39 27 301 a setup of an irradiation device can be learned about having a light source emitting UV-A and UV-B light, a two-piece light reflecting element (reflector), where the one light reflecting surface has absorbing, reflecting, or transmitting properties regarding UV-B, and the other is designed to have absorbing, reflecting, or transmitting properties regarding UV-A, and the two surfaces can be moved relative to each other. By changing the relative positions of the two surfaces, the UV-A/UV-B ratio in the emitted lamp light can be altered. A drawback of the known lamps is that by using one light source they are capable to irradiate only from one side, in a concentrated manner, but in case of application of several or a great number of light sources the energy consumption of the apparatus will be extremely high.

It is the purpose of the present invention to grant a tanning lamp arrangement which by applying a small number of light sources, under low acquisition and maintenance costs provides an uniform irradiation of bodies of different size, with a light having a spectrum similar to sunshine, whereas the harmful irradiation components can be filtered off and/or absorbed.

Surprisingly we have found that a person, not exposed to the direct effect of UV light, staying in a room with lime-washed walls, in the neighborhood of one single UV light source (low-, medium- or high-pressure gas-filled discharge lamp operating with arc light) on those regions of the body (nape, shoulder) where the light reflected from the walls reached him, has uniformly been tanned following a few number of “expositions”. The even tanning effect of the diffuse UV light reflected from the wall in this setup was then consequently tried out on test persons, who as having different skin types, showed different sensitivities against sunburn, in accordance with the aim of the scheduled practical application.

From this we have arrived at the conclusion, that the UV light—by inserting in its way (reflecting, absorbing, filtrating) optical elements of appropriate surface that are able to weaken the high power radiation coming from the UV light source by scattering and absorption, capable of filtrating off the harmful rays from it—may also be suitable to solar-cosmetic or phototerapeutic purposes. It is important that no harmful radiation (e.g. UV-C) should reach the person treated, and that the indirect light of modified wawelength and intensity could exert its proper effect, respectively.

If we still possess reflectors of suitable material, mirrors reflecting UV light, then it is possible to concentrate the light of modified wawelength and intensity onto body parts of the individual person to be treated providing there a proper light flux to phototherapy.

The most general solution of the task set out by the invention is the following:

The tanning lamp arrangement according to the invention has at least one light source arranged for the exposure of one side of the body, placed along a particular line, essentially parallel with the longitudinal axis of the body, and further the light reflecting optical elements are designed as a first light reflecting surface producing an uniformly scattered light for the exposure of the body. In order to expose the whole body surface, the body may rotate, or can be lightened from multiple side, simultaneously. In case of such an arrangement the body exposed to light is placed into a space that is “saturated” with scattered diffuse light. Contrary to prior art, the first light reflecting surface does not enclose the room around the light source only, but being rather spacious, in addition to the light source it surrounds the exposed body, too, so that the latter also can take up room in the space limited by the light reflecting surface. By the proper choice of the geometry of the light reflecting surface the light of a single source or that of a small number of light sources can efficiently be directed towards the body exposed, or may effectively be converted to diffuse light, filling up entirely the room around the body exposed.

In the most simple case the first light reflecting surface is a surface being provided by a carrier layer, for example, a coat of plaster, or a surface coated with a priming, that is covered by a top-layer, for example, by a lime-washed coating or by an other paint layer.

According to a preferred embodiment of the invention the first light reflecting surface is forming, at least partially, an elliptic surface, where the light source(s) is(are) essentially placed into a focal point of the elliptic. Such an arrangement will provide the utilization of the concentrated light emerging from the light source(s), as rays emerging from the light source placed into one focal point, will meet each other in the other focal point.

In case when the UV light source is emitting radiation in a spectral range, a part of which is harmful, or is not wanted to be used, it would be expedient if there is a radiation decreasing surface placed between the UV light source and the exposed body, which, for example, may be a shielding surface or a light filter. By such an arrangement it can be prevented a direct access of the light from the light source to the exposed body from happening, and thus the body will be exposed only to a light of properly modified spectral characteristics.

In the tanning lamp arrangement according to the invention the radiation reducing surface placed between the UV light source and the exposed body is, for example, a light filtering insertion, which covers the space between the light reflecting surfaces fully, if needed. In an other execution example the radiation reducing surface placed between the UV light source and the exposed body, is a shielding surface, which fully screens the direct radiation emerging from the light source.

In an other possible embodiment of the invention the radiation reducing surface placed between the light source(s) and the exposed body as a mirror-like second light reflecting surface, which covers optionally only a part of the room inside the first light reflecting surface. The mirror is preferably a flat, a convex, or a concave mirror of metal surface, for instance, a mirror of aluminium surface. In case of more than one light source, an arrangement may also be expedient, where each light source has an own reflector of convex or concave surface. In this case a light filter may be placed between the second light reflecting surface and the first light reflecting surface. By applying the light filter the spectrum of the light emitted from the light source can be modified according to requirements.

An other buildup may also be preferable, where that part of the first light reflecting surface which is covered by the second light reflecting surface is formed as a light reflecting mirror surface. Such an arrangement improves the utilization of light of the light sources.

In the tanning lamp arrangement according to the invention the first light reflecting surface is formed as an uniformly reflecting surface throughout the entire emission spectrum of the UV light source. In this case spectrum of the illuminating light may be regulated by the appropriate choice of the UV light source. As light source, discharge lamps of low, medium and high pressure can be taken into consideration, which have powerful enough emission also in the UV-A and UV-B spectral ranges. In the tanning lamp arrangement according to the invention the first light reflecting wall suitably has a carrier surface, a binding material layer, and an UV light reflecting material embedded in the binding material layer or applied on its surface. As a carrier surface will suit any surface of a material that has appropriate solidity, stiffness and form stability. Such material can be for example a brick wall, a plasterboard wall, or a wall based on metal, wood, paper, glass or plastic, or any combination thereof.

According to another possible embodiment, the first light reflecting surface consists of two bordering surfaces, and a material reflecting light in the UV region, arranged between the bordering surfaces, where the inner surface is transmitting or filtrating light in the UV region. These surfaces may be build up also from long and narrow cells that are fixed to each other.

The UV light reflecting material embedded in the binding material layer, or applied on it, or placed between the surfaces, or in the cells, exhibits a high reflexion factor, optionally a high refractive index, and a minimum light absorbance in the UV range, contains, for example, at least one material selected from the group of Al₂O₃, CaO, SiO₂, MgO, ZrO₂, Ta₂O₅, TiO₂, MgCO₃, CaCO₃. The light reflexion and the light absorbance spectra of the given materials are essentially uniform in the visible and in the UV spectral ranges.

It also proved to be advantageous if the particle size of the light reflecting material is smaller than the wawelength of the light to be reflected. In case of UV light this value can be less than 400 nm. In the UV region, this particle size range ensures the highest light reflective index, which results in enhanced utilization of light.

Another favourable embodiment may be, where the wall forming the first light reflecting surface is made of metal, for example of aluminium, which contains lamellas scattering light in every direction. According to another possible embodiment on the surface of one side of the elliptic wall that forms the first light reflecting surface, a light reflecting metal layer is developed. Such a wall surface may be made for example from an UV translucent plastic, while the metal layer can be for example an aluminium layer made by evaporization. In order to achieve a diffuse reflexion of light the surface of the translucent plastic, opposite to the metal layer, can be made rough by sand-blasting.

In the tanning lamp arrangement according to the invention the first light reflecting surface may also be designed so that it would exhibit enhanced reflective properties against the UV-A and/or UV-B spectral ranges. To achieve this, the surface may contain, for example, a phosphor compound as a light reflective material radiating light within the UV-A and/or UV-B ranges.

### SHORT DESCRIPTION OF DRAWINGS

Favourable solutions according to the invention are disclosed by making use of the figures. The particular embodiments, the different variations shown here, serve only aiming at to illustrate the invention but it is not limiting its scope any way, whatsoever.

FIG. 1 shows a top-view outline of the full-size tanning lamp arrangement according to the first example of the invention, the

FIG. 2 shows a top-view outline of the full-size tanning lamp arrangement according to the second example of the invention, the

FIG. 3 shows a top-view outline of the full-size tanning lamp arrangement according to the third example of the invention, the

FIG. 4 shows a top-view outline of the half-size tanning lamp arrangement according to the fourth example of the invention, the

FIG. 5 shows a top-view outline of the reduced size tanning lamp arrangement according to the fifth example of the invention, the

FIG. 6 shows a top-view outline of the first version of the first light reflecting surface applied in the tanning lamp arrangement according to the invention, the

FIG. 7 shows a top-view outline of the second version of the first light reflecting surface applied in the tanning lamp arrangement according to the invention, the

FIG. 8 shows a top-view outline of the third version of the first light reflecting surface applied in the tanning lamp arrangement according to the invention, the

FIG. 9 shows a top-view outline of the fourth version of the first light reflecting surface applied in the tanning lamp arrangement according to the invention, the

FIG. 10 shows a front-view outline of a version of the light source applied in the tanning lamp arrangement according to the invention.

### DISCLOSURE OF EXAMPLES OF PREFERRED EMBODIMENTS OF THE INVENTION

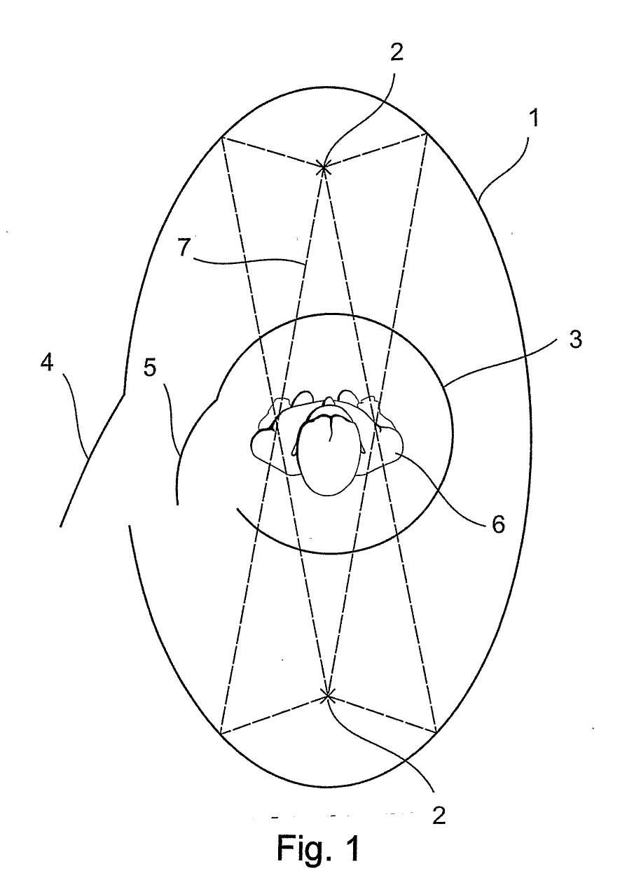

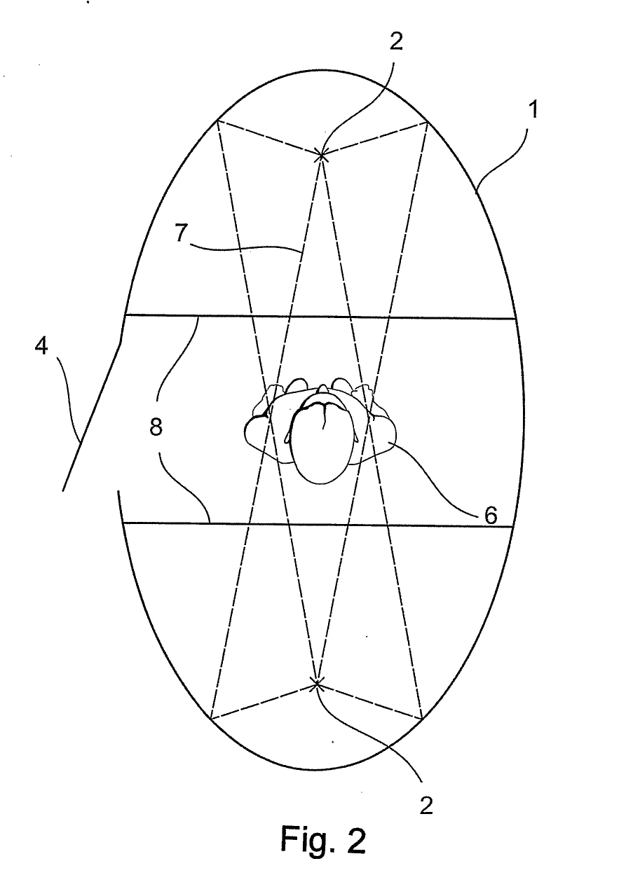

In case of the tanning lamp arrangement seen on FIGS. 1 and 2, there are two UV light sources placed into a space limited by a 1 light reflecting surface, where the 2 light source is a light source emitting radiation of suitable power also in the UV-A and UV-B spectral ranges. At the example shown on the Figure drawing the 6 exposed body is placed also in the space limited by the 1 light reflecting surface.

The 1 first light reflecting surface is formed as being a light reflecting surface that produces diffuse light illuminating the space around the exposed body, evenly. In the most simple case the first light reflecting surface is a plastered wall surface, on which there is optionally a lime-washed covering layer or a layer containing an other material reflecting light in the UV range. In the example showed herein the first light reflecting surface is basically an inner mantle of an elliptic-based cylinder, where the light sources are placed actually in one or the other focal points of the ellipse. In such an arrangement lighting with an uniform diffuse light is achievable in the whole room limited by the first light reflecting surface, by using a minimum number of light sources of suitable power. In a tanning lamp arrangement according to the invention there is at least one light source, placed along a particular line, essentially parallel with the vertical axis of the body, arranged for the exposure of one side of the body. In order to expose the whole body surface, the body may rotate, or can be illuminated from multiple side, simultaneously.

In between the light sources and the exposed body there is a 3 cabin of essentially cylindrical shape, which serves as a recipient room for the exposed body, and provides a suitable distance from the light sources to the exposed body. The wall of the 3 cabin is fully transparent, that is translucent in the visible and UV spectral ranges, or fully absorbent, or selectively absorbent in given spectral ranges, thus of having a light filtering effect. When a transparent wall cabin is applied the spectrum of the light irradiating the exposed object can be secured by the appropriate choice of the light source. By using a cabin wall of light filtrating effect any spectral range can be filtered off, or be absorbed from the spectrum of the light source. Since the solarium 3 cabin is of the same distance from both light sources, it provides an irradiation of the same intensity, therefore the same tanning or phototherapeutic effect from both directions. To provide access to the solarium room there are 4, 5 doors on the 3 cabin, and on the 1 wall possessing the first light reflecting surface, respectively. In case of the example shown, both 4, 5 doors are opening outwards, other solutions are however imaginable, too, where both 4, 5 doors are opening inwards, or the one 4 door opens outwards, whereas the other 5 door opens inwards. In case of the example shown on FIG. 1 both the 4, 5 doors are of bent surface and thus fitting to the elliptic outer wall, as well as to the cylindrical inner wall.

On FIG. 2, in place of the cabin, there are 8 room partitioning walls put on that can equally be fully transparent, or of uniformly light absorbing, or of selectively light absorbing, i.e. of light filtering effect. In case of a transparent 8 room partitioning wall the spectrum of the irradiating light can properly be secured via appropriate selection of the 2 light source. By a partition wall of a light filtrating effect, optional UV ranges can be filtered off, or absorbed from the UV spectrum of the 2 light source.

The space inside the 1 light reflecting surface is fully enclosed by the 8 room partitioning walls. In this arrangement it is only one 4 door required on the outer elliptic wall. In case of the example seen on the drawing, the 4 door is flat and opens outward, but it may equally be a door opening inward, too. Moreover each door may also be designed as a sliding door, which is especially preferred for its room saving feature. The path of the light emerging from the 2 light source, and passing forth after having been reflected from the light reflecting wall is marked with 7 dotted line. As it is clearly seen on FIGS. 1 and 2, the light beams coming from the 2 light source(s) placed into the focal point of the elliptic, reflecting from the wall, are meeting each other inside the 1 light reflecting surface, in the other focal point. The light reflecting surface is designed so that the reflected light yields a diffuse irradiation, which makes the irradiation even more uniform and homogeneous. Dimensions of the elliptic base-board are determined by the major axis and the minor axis of the elliptic. For buildup of a standing type solarium, diameter of the cabin is possible to be varied between 60-80 cm, while the minor axis of the elliptic is possible to be varied between 90-150 cm, and the major axis of the elliptic is possible to be varied between 100-300 cm.

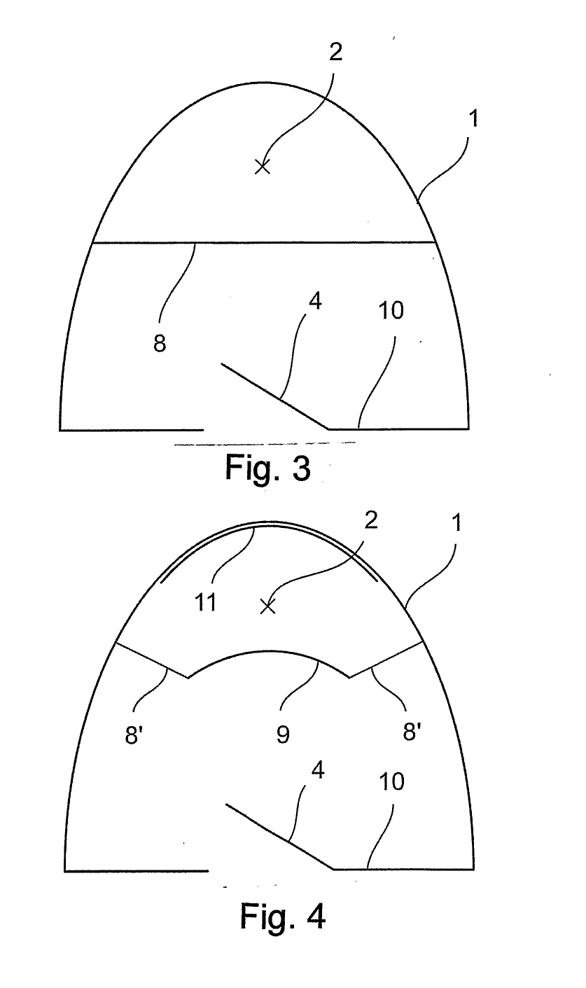

On the FIGS. 3 and 4, a tanning lamp arrangement is seen, where the 1 light reflecting surface gives a cylindrical surface the base of which is actually an elliptic piece cut, in the case in question, a half elliptic. The setup so designed requires only half space, and therefore it fits well into smaller rooms too. Since at such arrangements the 2 light source can be placed only to one side, the exposed object or body should be turned around, or be rotated during the irradiation. In case of these execution examples the 2 light source is placed essentially the same way in the focal point of the 1 light reflecting surface, and formation of the surface is also corresponding to that demonstrated in FIGS. 1 and 2. On FIG. 3, similarly to FIG. 2, there is a transparent or light-absorbing room partitioning wall placed in between the 2 light source and the exposed body. In case of a transparent 8 room partitioning wall the spectrum of the light irradiating the object may properly be ensured via the suitable selection of the 2 light source. By using a partition wall of light filtrating effect, UV regions can optionally be filtered off, or absorbed from the UV spectrum of the 2 light source. A filter like this has radiation reducing effect, so the energy of the light sources is absorbed to different extent, depending on the wawelength of the light.

Therefore, it can be secured that the intensity of rays having damaging effect harmful e.g. to the human skin, be minimal. When a phototherapy treatment is aimed at, by choosing a filter of appropriate characteristics the desired therapeutic effects may be achieved. In case of this example the suntanning room is delimited also by the separation wall of light filtrating effect, on the side from the direction of the lamp. On the other sides, the lateral walls of the suntanning room are delimited/bordered by the elliptical 1 light reflecting surface and by a flat wall, on which also a 4 door is placed enabling to enter or leave, therethrough. Although according to the examples shown, the 4 door opens inwards, but a door opening outwards may equally be appropriate. In the example shown on FIG. 4, there is a 9 shielding surface, placed between the 2 light source and the exposed body, having a radiation reducing effect, in addition supporting the 2 light source, which entirely prevents the direct light from getting across to the direction of the exposed body. The 9 shielding surface is covering only a part of the room inside the 1 first light reflecting surface. It is also possible that a 8′ filter be placed between the 9 shielding surface and the 1 first light reflecting surface, if needed. The radiation reducing surface placed between the 2 light source and the exposed body, may be a mirror-like second light reflecting surface, too, which would increase the light utilization of the light sources. In order to step up further the light utilization, that part of the 1 first light reflecting surface, which is covered by the 9 shielding surface, may also be designed as a 11 mirror-like light reflecting surface.

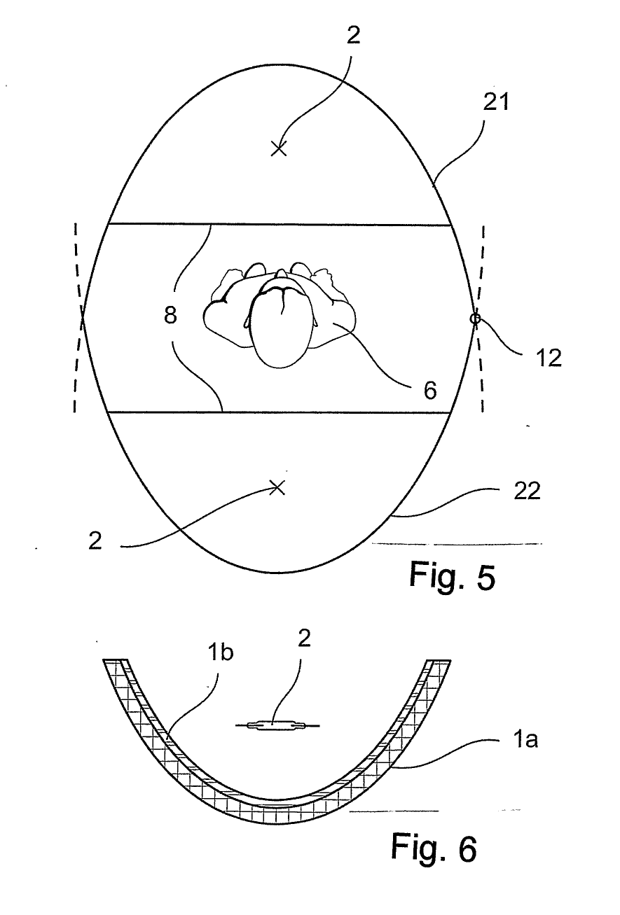

On the FIG. 5 an additional option is seen for reducing the room requirement. In this example the elliptical 1 light reflecting wall, according to FIG. 2, has been cut through in the middle, and the open endings of the wall have been shortened inasmuch as it was marked by the dotted lines. By fitting together the remaining parts such light reflecting elliptical surface pieces have been obtained, at which the two focal points are coming nearer to each other, and the length of the elliptic floorspace has been shortened substantially, while its width has only changed to a small extent, becoming smaller. According to this solution, as compared to the execution example pursuant to FIG. 2, the size of the room for suntanning remained essentially the same, whereas the full length of the tanning lamp arrangement has become substantially smaller. The buildup of the 1 light reflecting surface, the placing of the 8 shielding or light filtrating surfaces is to be applied according to any of the variants demonstrated on FIGS. 1 to 4. There is no door displayed on FIG. 5, it can however be built in according to a variation pursuant to FIG. 1 or 2. The execution examples shown above are equally fitting to design a standing as well as a lying version of the solarium setup. In case of a lying version it is expedient to carry on with the buildup of the solution according to FIG. 5 so, that one from among the two 21, 22 elliptical parts would form a fixed 22 lower part, while the other one forms a 21 upper part that can be turned away or opened up, relative to the fixed part. The 2 light sources are placed here again in the focal points of the elliptic parts. There is a possibility, even in case of the lying version, to apply only one elliptic light reflecting surface, where the upper part that can be opened up forms an elliptic light reflecting surface, while the fixed lower part is a flat surface, providing a surface to lie down to for the person to be treated. Similarly, an other setup is conceivable, where the fixed lower part forms the elliptic light reflecting surface. In such a case the person to be treated is lying on a plain surface that is translucent or of light filtering effect in the UV region.

FIG. 6 shows a part of the first light reflecting surface of the tanning lamp arrangement according to the invention. In case of the buildup shown on the drawing, the first light reflecting surface has a 1a carrier surface, a 1b binding material layer, and a material reflecting light in the UV range, that is embedded in the binding material layer or applied on its surface. Provided a 2 light source of appropriately chosen spectrum and/or filters are applied, the first light reflecting surface may be designed so that it would function as an uniformly reflecting surface throughout the entire irradiation spectrum of the UV light source. To obtain such a surface, the light reflecting material would have a high reflexion factor, possess optionally a high refractive index, and a minimal light absorption in the UV range, that contains at least one material selected for example from the group of Al₂O₃, CaO, SiO₂, MgO, ZrO₂, Ta₂O₅, TiO₂, MgCO₃, CaCO₃ (carbonates), in an optional mixing ratio. In order to achieve a maximal light scattering effect, it is worth choosing the particle size of the light reflecting material to be less than the wawelength of the light to be reflected, for instance, to less than 400 nm. According to an other possible version, the first light reflecting surface is formed as a surface of enhanced reflectivity in the UV-A and/or UV-B spectral regions towards the radiation emitted by the UV light source. To provide this the light reflecting surface contains a phosphor compound as a light reflecting material emitting radiation, for example, in the UV-A and/or UV-B spectral regions.

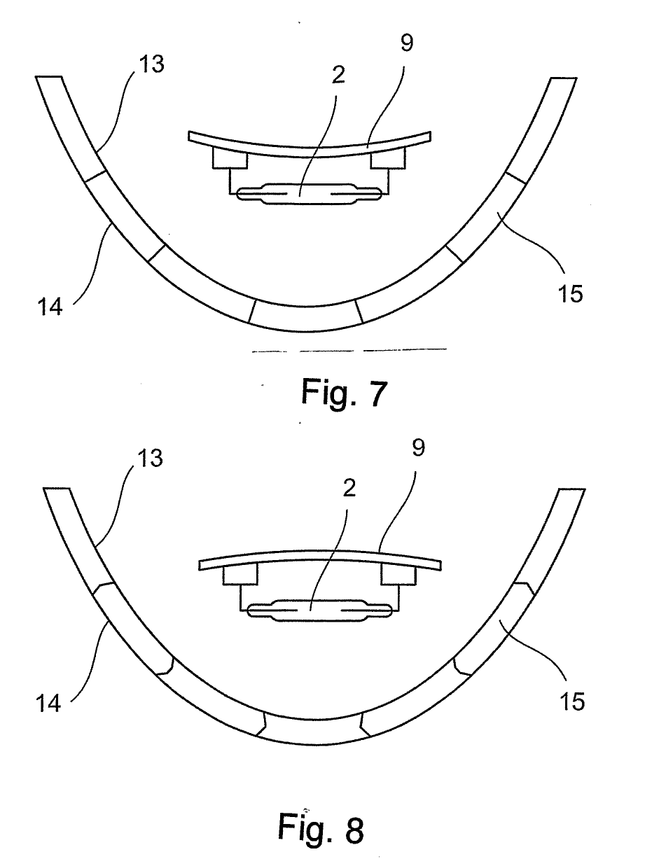

FIGS. 7 to 9 are presenting such variations of embodiment of the first light reflecting surface, where the light reflecting material is arranged in between two 13, 14 bordering surfaces, and where the 13 inner surface is transmitting light in the UV range, or possessing a light filtrating effect, if needed. Similarly to the example shown on FIG. 6, the light reflecting material arranged between the bordering walls has a high reflexion factor in the UV range, possess optionally a high refractive index and a minimal light absorption, which contains at least one material selected for example from the group of Al₂O₃, CaO, SiO₂, MgO, ZrO₂, Ta₂O₅, TiO₂, MgCO₃, CaCO₃. In case of several components their ratio is optional. In order to achieve maximal light scattering effect, it is worthwhile to select a particle size of the light reflecting material to be less than the wawelength of the light to be reflected, for instance, to less than 400 nm. In accordance with an other possible version, the first light reflecting surface is formed as a surface of enhanced reflectivity in the UV-A and/or UV-B spectral ranges towards the radiation emitted by the UV light source. To provide this the light reflecting surface contains a phosphor compound as a light reflecting material emitting radiation within the UV-A and/or UV-B spectral ranges.

In the example presented by FIG. 7, the elliptical light reflecting wall is divided to 15 wall elements of essentially the same width, which are determining space parts delimited by walls. From among the bordering walls the inner 13 bordering wall is made of a plastic having light transmitting or light filtrating properties in the UV region, the other walls are of light transmitting properties to preference, thus can also be of a material not transmitting light. The parts of the wall are fixed to each other by separable or unseparable bonds, optionally. As a separable bond, can for example, fastening with screws, while as an unseparable bond, may for example, riveting or sticking be applied for. Such an arrangement increases the stiffness and the form keeping of the light reflecting wall elements, and prevents the light reflecting material placed in between the bordering surfaces from bulging out the bordering surfaces to an appreciable extent, and thus deforming the elliptic wall geometry.

On FIG. 8 a further variant of the setup according to FIG. 7 is seen, where some 15 wall elements of the elliptic light reflecting wall fit together by special surfaces. To facilitate an exact fitting, the abutting surfaces may form for example wedges. The special fitting surfaces may be identical, but they can also be different, according to the execution example shown on the drawing. In the case presented here the central wall possess symmetric fitting surfaces, while the other walls have asymmetric ones. With a setup like this determining of the position of the particular wall elements by encoded fitting parts can easily be ensured what substantially simplifies assembling, and precludes the possibility of interchange during assembly.

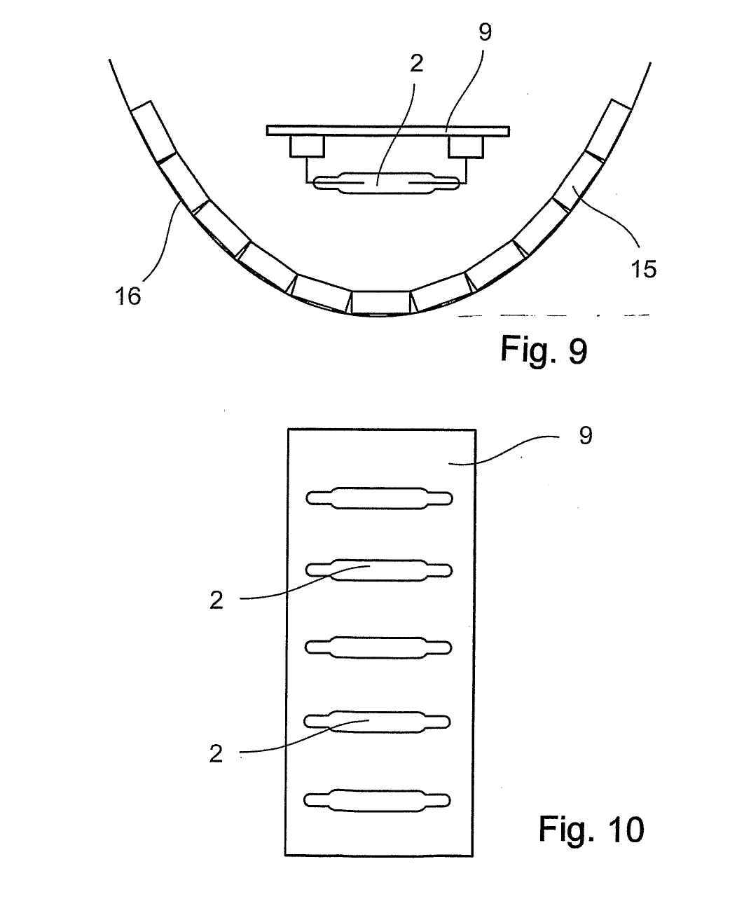

On FIG. 9 a further embodiment of the light reflecting wall is seen according again to FIG. 7. In this version the 15 wall elements have rectangular cross-sections and are totally identical, consequently any of these wall elements can be placed anywhere along the elliptic light reflecting wall. In this case the elliptic form is assured by an elliptic 16 supporting wall or by a line of stiffening ribs, being in a plane essentially perpendicular to the wall, which the particular wall elements are fixed to. The 15 wall elements can be so long that they cover the light reflecting wall entirely, but they may also be shorter, whereas several wall elements are connected to each other lengthwise in order to cover the full length. The longitudinal fitting does not appear on the top-view drawing. Such a buildup will simplify both the manufacturing of the wall elements, and their assembly, substantially. In case of not too wide (e.g. of 5-10 cm length) wall elements, the elliptical surface can be established by suitable accuracy and good approximation, in accordance with the invention.

In case of not too wide (e.g. of 5-10 cm length) wall elements, the elliptical surface can be formed by an appropriate accuracy and with good approximation, according to the invention.

In order to provide an uniform illumination the 2 light source actually placed in the focal point of the elliptic 1 light reflecting wall can be a single, long, tubular light source (for instance a medium-pressure discharge lamp) or a composite light source combined from several smaller size light sources, where the particular light sources are distributed along a common axis. The light source(s) is (are) fixed on such a framework, which at the same time is also serving as a 9 shielding surface. In addition, an other framework is conceivable too, which is fixed to the 1 light reflecting surface. The shielding surface may be flat (FIG. 9), convex (FIG. 7), or concave (FIG. 8). When applying several light sources, in the own spectral range of the particular light source, there can be a concave or convex reflector surface, directing the light of the given source, focused or diffusely, to the first light reflecting surface. FIG. 10 shows a possible arrangement of the light sources. In case of the example seen on the Figure, a front-view of the light source, placed into one focal point of the elliptic light reflecting wall, can be seen, which in itself constitutes a composite light source assembled from a number of primary 2 light sources. In case of the executive example shown on the Figure the light sources are high-pressure discharge lamps, the optical centers of which are aligned essentially in a straight line, while the latter is in the top-view positioned actually in the focal point of the elliptic light reflecting surface (FIGS. 7 to 9). In the execution example shown here, the longitudinal axis of the particular 2 light sources is essentially perpendicular to the longitudinal axis of the 9 framework. Applying standing type solariums and high-pressure discharge lamps, this setup is advantageous, since in case of using high-pressure discharge lamps the manufacturer recommends a horizontal building in. In case of lying type solariums there is also an option already, that the longitudinal axis of the particular light sources be essentially parallel with the longitudinal axis of the 9 framework. Applying standing type solariums and high-pressure discharge lamps, this setup is advantageous, because in the case of using high-pressure discharge lamps the manufacturer recommends a horizontal building in. In case of lying type solariums there is also an option already that the longitudinal axis of the particular light sources be essentially parallel with the longitudinal axis of the 9 framework. The row of lamps seen on the Figure may extend to the whole size of height of the first light reflecting surface, but it is also possible that, by applying fewer lamps, only a part thereof is taken up. In the case of applying a lamp or lamp row being shorter than the height of the first light reflecting wall, by using an appropriate machinery, care must be taken about the moving of the lamp or the row of lamps in vertical direction. In order to achieve an uniform illumination it may be expedient to accomplish moving with a varying speed, whereas its speed is greater in the middle range, while in the side ranges (namely above and under) it is less.

Another favourable embodiment may be established when the first light reflecting surface is made of a metal, for example of aluminium, which contains lamellas scattering light in every directions.

The invention has been disclosed based on the exemplary embodiments represented by the drawings, it does not mean however that the invention were restricted to any of the execution examples. It is obvious for an expert that a number of modifications, improvements and combinations of the exemplary embodiments demonstrated here is conceivable within the scope of protection of the invention, which is determined by the claims presented hereinbelow.

1. Tanning lamp arrangement, especially for suntanning the skin surface of the human body, having at least one UV light source radiating UV light with an appropriate intensity also in the UV-A and/or UV-B spectral ranges, and having optical elements reflecting the light of the light source(s), placed upon the side of the human body opposed to the light source(s), characterized by at least one light source arranged for the exposure of one side of the body, placed along a single line, essentially parallel with the longitudinal axis of the body, and further the light reflecting optical elements are configured as a first light reflecting surface with a single, substantially continuously curved surface for producing an uniformly scattered light for the exposure of the body.

2. Tanning lamp arrangement according to claim 1, characterized by that the first light reflecting surface is, at least partially, forming an elliptic surface, where the light source(s) is (are) placed essentially in the focal point(s) of the elliptic.

3. Tanning lamp arrangement according to claim 1, characterized by that light filtering surface(s) are placed between the light source(s) and the exposed body.

4. Tanning lamp arrangement according to claim 1, characterized by that between the light source(s) and the exposed body there is a shielding surface which is designed as a framework supporting the light source(s).

5. Tanning lamp arrangement according to claim 1, characterized by that the radiation decreasing surface, placed between the light source(s) and the exposed body, is a second, mirror-like light reflecting surface.

6. Tanning lamp arrangement according to claim 5, characterized by that the second light reflecting surface, placed between the light source(s) and the exposed body, is extending only to a part of the space inside the first light reflecting surface.

7. Tanning lamp arrangement according to claim 6, characterized by that a light filter (8′) is placed between the second light reflecting surface, that is in between the light source(s) and the exposed body, and the first light reflecting surface.

8. Tanning lamp arrangement according to claim 7, characterized by that the part of the first light reflecting surface hidden by the second light reflecting surface, is formed as a mirror-like light reflecting surface.

9. Tanning lamp arrangement according to claim 1, characterized by that the first light reflecting surface is formed as an uniformly reflecting surface throughout the emission spectrum range of the UV light source.

10. Tanning lamp arrangement according to claim 9, characterized by that the first light reflecting surface is made from a metal reflecting in the UV spectrum range.

11. Tanning lamp arrangement according to claim 9, characterized by that the first light reflecting surface is formed as an elliptic wall made of an UV light transmitting plastic, of which one side is covered by a metal coating reflecting light in the UV range, whereas the other side is made rough by sand-blasting.

12. Tanning lamp arrangement according to claim 9, characterized by that the first light reflecting surface has a carrier surface, a binding material layer, and a material reflecting light in the UV range, that is embedded in the binding material layer or applied on its surface.

13. Tanning lamp arrangement according to claim 9, characterized by that the first light reflecting surface consists of two bordering surfaces and of an UV light reflecting material arranged between the two bordering surfaces, whereas the inner surface exhibits light transmitting or light filtrating properties in the UV range.

14. Tanning lamp arrangement according to claim 13, characterized by that the first light reflecting surface made up of two bordering surfaces are forming a closed space surrounded by long and narrow surface elements where the light reflecting material are placed in the closed space, and the surface elements forming the closed spaces are fixed to each other.

15. Tanning lamp arrangement according to any of the claims 12-14, characterized by that the light reflecting material exhibiting a high reflexion factor, optionally a high refractive index, and a minimum light absorbance in the UV range, contains at least one material selected from the group of AI203, CaO, Si02, MgO, Zr02, Ta205, Ti02, MgCO3, CaCO3.

16. Tanning lamp arrangement according to claim 1, characterized by that the first light reflecting surface s formed as a surface of enhanced reflecting properties concerning the radiation emitted by the UV light source in the UV-A and/or UV-B spectrum ranges.

17. Tanning lamp arrangement according to claim 16, characterized by that the light reflecting surface contains phosphor radiating in UV-A and/or UV-B ranges as light reflecting material.
